(19) United States
(12) Patent Application Publication
(10) Pub. No.: US 2007/0162051 A1
(43) Pub. Date: Jul. 12, 2007

(54) Electrode implant tool

(75) Inventors: Joseph L. Calderon, Porter Ranch, CA (US); Ross Davis, Melbourne Beach, FL (US); Alfredo Araujo, Sylmar, CA (US); Delta Mishler, Burbank, CA (US)

Correspondence Address: ALFRED E. MANN FOUNDATION FOR;SCIENTIFIC RESEARCH, PO BOX 905, SANTA CLARITA, CA, 91380, US

(73) Assignee: Alfred E. Mann Foundation for Scientific Research, Santa Clarita, CA (US)

(21) Appl. No.: 11/395,564

(22) Filed: Mar. 31, 2006

Related U.S. Application Data
(60) Provisional application No. 60/759,219, filed on Jan. 12, 2006.

(51) Int. Cl. A61B 19/00

(52) U.S. Cl. 606/129; 607/108

(57) ABSTRACT

The invention is a hollow tube electrode inserter for inserting an electrode such as a Memberg electrode in living tissue. The inserter has a longitudinal slot that accepts the electrode into the hollow center of the tube inserter. The slot is offset at least once forming one or more retainer tabs that assure retention of the electrode in the inserter during insertion of the electrode in living tissue.

### CROSS REFERENCE TO RELATED APPLICATION

This application claims the benefit of U.S. Provisional Application Ser. No. 60/759,219, filed on Jan. 12, 2006.

### BACKGROUND OF THE INVENTION

1. Field of the Invention

This invention relates to a device for placement of a stimulator or sensor in living tissue.

2. Description of Related Art Including Information Disclosed Under 37 CFR 1.97 and 1.98

Microstimulators are small, implantable electrical devices that pass a signal to living tissue in order to elicit a response from a nerve or muscle. Microsensors are similar electrical devices except that they detect electrical and other signals that are generated by living tissue. The term microstimulator is intended to apply equally to both microstimulators and microsensors. The use of microstimulators or microsensors which are implanted in living tissue to stimulate a muscle function by either stimulating a nerve or the muscle itself are well known. The microstimulators receive power and control signals by inductive coupling of magnetic fields generated by an extracorporeal antenna rather than requiring any electrical leads. See for example, U.S. Pat. Nos. 5,193,539; 5,193,540; 5,324,316; 5,405,367; 6,175,764; 6,181,965; 6,185,452; 6,185,455; 6,208,894; 6,214,032; and 6,315,721, each of which is incorporated in its entirety by reference herein. These microstimulators are particularly advantageous because they can be manufactured inexpensively and can be implanted non-surgically by injection. Additionally, each implanted microstimulator can be commanded, at will, to produce a well-localized electrical current pulse of a prescribed magnitude, duration and/or repetition rate sufficient to cause a smoothly graded contraction of the muscle in which the microstimulator is implanted.

While primarily designed to reanimate muscles so that they can carry out purposeful movements such as locomotion, the low cost, simplicity, safety and ease of implantation of these microstimulators suggests that they may additionally be used to conduct a broader range of therapies in which increased muscle strength, increased muscle fatigue resistance and/or increased muscle physical bulk are desirable; such as therapies directed to muscle disorders. For example, electrical stimulation of an immobilized muscle in a casted limb may be used to elicit isometric muscle contractions that prevent atrophy of the muscle for the duration of the casting period and facilitate rehabilitation after the cast is removed. Similarly, repeated activation of microstimulators injected into the shoulder muscles of patients suffering from stroke enable the paretic muscles to retain or develop bulk and tone, thus helping to offset the tendency for such patients to develop subluxation at the shoulder joint. Use of microstimulators to condition perineal muscles increases the bulk and strength of the musculature in order to maximize its ability to prevent urinary or fecal incontinence. See for example, U.S. Pat. No. 6,061,596, which is incorporated in its entirety by reference herein.

Microstimulators, as exemplified by the BION® of Advanced Bionics Corporation, are typically elongated devices with metallic electrodes at each end that deliver electrical current to the immediately surrounding living tissues. The microelectronic circuitry and inductive coils that control the electrical current applied to the electrodes are protected from the body fluids by a hermetically sealed capsule. This capsule is typically made of a rigid dielectric material, such as glass or ceramic, which transmits magnetic fields but is impermeable to water.

Often, while placing the miniature microstimulator in living tissue, the orientation of the microstimulator changes slightly such that the microstimulator is not in fact in electrical contact with the nerve, requiring reorientation of the microstimulator. The microstimulator may move at any point in the surgical implantation procedure. If the microstimulator has moved, it may be at a significant distance from the nerve that is to be stimulated. Consequently, more energy is needed from the microstimulator to stimulate the nerve, unless the microstimulator is repositioned closer to the nerve. While such microstimulators may be injected, the actual placement requires first locating the desired end point near the nerve or muscle. The known method of placement involves locating the nerve with an electric probe, placing a hollow implantation tool over the electric probe and removing the electric probe to allow the miniature microstimulator to be passed down the length of the hollow implantation tool. The implantation tool is then removed, leaving the microstimulator implanted at or near the desired location. If there is a problem with the function or location of the microstimulator, then additional surgery must be performed to remove or relocate the microstimulator, imposing risk, discomfort and potential tissue damage to the patient.

Using a known implantation tool, as disclosed in U.S. Pat. No. 6,214,032, to implant a microstimulator, may lead to the device being located remotely from the desired nerve. In this approach, an electrically stimulating trocar is first used to locate the desired nerve. The trocar is removed, after a cannula is slid along the trocar to be next to the nerve. Then the microstimulator is placed next to the nerve by inserting the microstimulator into the cannula and pushing the microstimulator to the end of the cannula, where it is ejected and is left behind, after the cannula is removed. The problem is that once the electrically stimulating trocar is removed, there is no way to detect movement of the cannula. Thus, the microstimulator may be left some distance from the desired location, as was determined by the stimulating trocar. This displacement from the optimum stimulating site unacceptably increases the power requirements and diminishes the battery life of the microstimulator.

Therefore, it is desired to have a method of implantation that ensures that the microstimulator is functioning properly and is implanted in an optimum position prior to removing the implantation tools that are utilized during surgery to place the microstimulator.

### BRIEF DESCRIPTION OF THE SEVERAL VIEWS OF THE DRAWINGS

FIG. 1 schematically depicts a perspective view of the electrode implant device with one retainer tab and one offset slot showing the electrode inserted inside the hollow tube.

FIG. 2 schematically depicts a perspective view of the electrode implant device with two retainer tabs and two offset slots showing the electrode inserted inside the hollow tube.

### DETAILED DESCRIPTION OF THE INVENTION

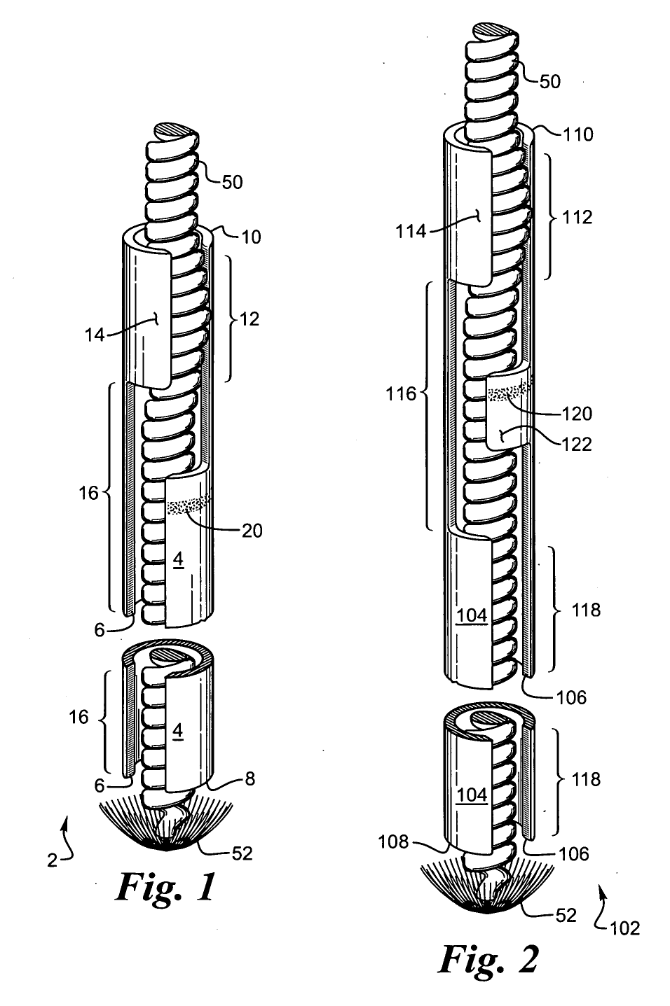

An insertion tool 2 is presented generally in FIG. 1 which comprises a hollow tube 4 having a thin wall 6. A longitudinal offset slot 12 begins at the proximal end 10 of the hollow tube 4 and extends with an approximately constant width longitudinally along the wall 6. The offset slot 12 is defined by a retainer tab 14, said offset slot 12 accepting an electrode 50, where in a preferred embodiment electrode 50 is a Memberg electrode, as is known in the art, having a distal end 52 that is designed to aid in retention of the Memberg electrode 50 after insertion in living tissue.

Said offset slot 12 jogs in an interrupted fashion to form a continuous longitudinal opening in the wall 6, defined as a retainer slot 16, extending to a distal end 8 of said tool 2. The electrode 50 is urged into said retainer slot 16 and into said offset slot 12, being retained in that position by retainer tab 14.

The insertion tool 2 has a depth mark 20 on the outside of the tool 2 which provides a visual reference to the desired depth of penetration of the tool 2 into living tissue.

The insertion tool 2 enables a surgeon to insert the electrode more easily than with known insertion tools, which have proven to be cumbersome and which require the use of two hands to retain or replace the electrode in the insertion tool during implantation.

An alternate embodiment of the insertion tool 102 is presented in FIG. 2. To assure better retention of the electrode 50 a second retainer slot 118 is added. The insertion tool 102 comprises a hollow tube 104 having a thin wall 106. A longitudinal offset slot 112 begins at the proximal end 110 of the hollow tube 104 and extends with an approximately constant width longitudinally along the wall 106, jogging to form retainer slot 116. The offset slot 112 is defined by a second retainer tab 114 and a center retainer tab 122, which keep the electrode 50 in the hollow tube during implantation.

Said offset slot 112 jogs in an interrupted fashion to form a continuous longitudinal opening in the wall 106. The retainer slot 116 jogs to form second retainer slot 118 which extends to the distal end 108 of hollow tube 104. The insertion tool 102 has a depth mark 120 on the outside of the tool 102 as a visual reference to the desired depth of penetration of the tool 102 into living tissue.

What is claimed is:

1. An electrode holding device, comprising:
a hollow tube defining a longitudinal offset slot for accepting an electrode to be implanted and a retainer slot for accepting the electrode; and
an integral hollow tube retainer tab to retain the electrode in said tube.

2. The electrode holding device according to claim 1, further comprising a second retainer slot for securing the electrode in said hollow tube.

3. The electrode holding device according to claim 1, further comprising a depth mark on said hollow tube to define position of said holding device.

4. An electrode holding device, comprising:
a hollow tube having longitudinal slots of sufficient length for receiving the electrode through the slots to position the electrode within said hollow tube; and
at least one retainer tab positioned along the length of the tube intermediate the slots and dimensioned to permit entry of the electrode into the tube and to maintain the electrode in the hollow tube, once the electrode is positioned therein.

5. The electrode holding device according to claim 4, further comprising a second retainer slot for securing the electrode in said hollow tube.

6. The electrode holding device according to claim 4, further comprising a depth mark on said hollow tube to define position of said holding device.
